(19) United States
(12) Patent Application Publication
(10) Pub. No.: US 2025/0271410 A1
(43) Pub. Date: Aug. 28, 2025

(54) SKIN PORE MIMIC AND METHOD FOR EVALUATING THE SUSTAINABILITY OF MAKEUP FILM USING THE SAME

(71) Applicant: AMOREPACIFIC CORPORATION, Seoul (KR)

(72) Inventors: Chaeyeon SONG, Yongin-si (KR); Kyungsup HAN, Yongin-si (KR); Youngkyu HAN, Yongin-si (KR)

(73) Assignee: AMOREPACIFIC CORPORATION, Seoul (KR)

(21) Appl. No.: 19/056,078

(22) Filed: Feb. 18, 2025

(30) Foreign Application Priority Data
Feb. 26, 2024 (KR) 10-2024-0027477

(51) Int. Cl. G01N 33/00; G01N 19/00

(52) U.S. Cl. CPC G01N 33/0096; G01N 19/00

(57) ABSTRACT

A skin pore mimic, and more specifically, the skin pore mimic, according to one aspect of the present disclosure, includes a PDMS layer that includes polydimethylsiloxane and has a contact angle with water ranging from 90° to 110°, in which the PDMS layer includes a plurality of holes that penetrate through an upper surface of the PDMS layer and a lower surface of the PDMS layer, thereby exhibiting characteristics similar to real skin in terms of hydrophilicity, elasticity, and the like, and enabling the simulation of sebum being secreted through pores. The skin pore mimic, according to one aspect of the disclosure, allows for an objective evaluation of the sustainability of a test material with respect to sebum.

### CROSS REFERENCE TO RELATED APPLICATION

The present application claims priority to Korean Patent Application No. 10-2024 -0027477, filed Feb. 26, 2024, the entire contents of which are hereby incorporated by this reference.

### FIELD OF THE INVENTION

The present disclosure relates to a skin pore mimic and a method of evaluating the sustainability of a cosmetic film using the same.

### BACKGROUND

Recent studies have been conducted to evaluate the breakdown or sustainability of makeup. The sustainability evaluation of makeup measures the extent to which it may withstand for a long time without running or smudging over time. Over time, on the surface of a person's skin, sebum produced by sebaceous glands located in the dermis is secreted through the pores to the skin surface, which may affect a cosmetic film. Additionally, makeup may break or clump due to the movement of the face. Therefore, to more accurately evaluate the breakdown or sustainability of makeup, it is necessary to consider the influence of oily components, such as sebum secreted from within the skin, while also observing changes caused by movement.

To evaluate the breakdown or sustainability of makeup, the following methods have primarily been used: i) applying a cosmetic composition to human skin and observing changes in the applied cosmetic composition, ii) applying sebum to artificial skin, then applying the cosmetic composition to be tested on top of the applied sebum to form a cosmetic film, and analyzing changes in the cosmetic film, iii) applying a cosmetic composition to the surface of a porous material, allowing sebum to randomly pass through the porous material, and analyzing changes in the applied cosmetic composition. These methods have the drawback of making it difficult to control the amount of sebum applied to the cosmetic composition, which is a test material, in a consistent manner, which hinders objective analysis.

### SUMMARY OF THE INVENTION

One aspect of the present disclosure is directed to providing a skin pore mimic that is capable of evaluating the sustainability of a cosmetic composition with respect to sebum.

One aspect of the present disclosure is directed to providing a method of evaluating sustainability that is capable of evaluating the sustainability of a cosmetic composition with respect to sebum.

According to one aspect of the present disclosure, there is provided a skin pore mimic, including a PDMS layer that includes polydimethylsiloxane (PDMS) and has a contact angle with water ranging from 90° to 110°, in which the PDMS layer includes a plurality of holes that penetrate through an upper surface of the PDMS layer and a lower surface of the PDMS layer.

According to one aspect of the present disclosure, there is provided a method of evaluating the sustainability of a test material, including: applying a test material to an upper surface of the PDMS layer of the skin pore mimic; and contacting a lower surface of the PDMS layer with artificial sebum.

The skin pore mimic according to one aspect of the present disclosure exhibits characteristics similar to real skin in terms of hydrophilicity, elasticity, structure, and the like, and can simulate the form in which sebum is secreted through pores.

By using the skin pore mimic and/or method of evaluating sustainability according to one aspect of the present disclosure, the sustainability of the test material with respect to oils and/or sebum can be objectively evaluated.

By using the skin pore mimic and/or method of evaluating sustainability according to one aspect of the present disclosure, the sustainability of a test material with respect to oils and/or sebum, as well as its sustainability under external forces, can be objectively evaluated.

### BRIEF DESCRIPTION OF THE DRAWINGS

FIG. 1 is a schematic view illustrating a skin pore mimic according to an embodiment.

FIG. 2 is a schematic view illustrating a skin pore mimic according to an embodiment.

FIG. 3 is a schematic view illustrating the process of evaluating the sustainability of a test material using a skin pore mimic according to an embodiment.

FIG. 4 is a schematic view illustrating the process of evaluating the sustainability of a test material using a skin pore mimic according to an embodiment.

FIG. 5 is a schematic view illustrating the structure of a hole of a skin pore mimic according to an embodiment. Rₜₒₚ represents the radius of the upper end cross-section of the hole, R_{bot} represents the radius of the lower end cross-section of the hole, and H represents the height of the hole.

FIG. 6A is a schematic side view illustrating a skin pore mimic with two PDMS layers according to an embodiment. FIG. 6B is a side-view photograph of a skin pore mimic with two PDMS layers according to an embodiment.

FIG. 7A to FIG. 7C illustrate that a skin pore mimic, according to an embodiment, exhibits hydrophilicity similar to that of actual human skin. FIG. 7A illustrates the contact angle of water on actual human skin, and FIG. 7B illustrates the contact angle of water on the skin pore mimic according to an embodiment. FIG. 7C illustrates the contact angle with water according to the content of the surfactant mixed with PDMS.

FIG. 8A to FIG. 8D illustrate the results of verifying the sebum secretion performance of a skin pore mimic according to an embodiment. FIG. 8A is a photograph of the upper surface of the skin pore mimic before the application of artificial sebum. FIG. 8B is a photograph of the lower surface of the skin pore mimic before the application of artificial sebum. FIG. 8C is a side-view photograph of the skin pore mimic before the application of artificial sebum. FIG. 8D is a photograph of the upper surface of the skin pore mimic after the application of artificial sebum.

FIG. 9A to FIG. 9B illustrate the process of evaluating the sustainability of a test material with respect to sebum, wherein the test material is applied to the upper surface of the skin pore mimic according to an embodiment. The left photograph of FIG. 9A is a photograph of a skin pore mimic according to an embodiment. The right photograph of FIG. 9A illustrates a state where a test material (makeup cosmetic composition) is applied to the top surface of the skin pore mimic according to an embodiment. FIG. 9B illustrates the process of applying a test material (makeup cosmetic composition) to the top surface of the skin pore mimic according to an embodiment, then immersing the mimic with the applied test material in a composition including artificial sebum to evaluate the sustainability of the test material with respect to sebum.

FIG. 10 illustrates the results of evaluating the sustainability of a test material (cushion foundation) with respect to sebum using a skin pore mimic according to an embodiment. N01 represents the experimental result of applying a first cushion foundation with weak sustainability to the skin pore mimic, N02 represents the experimental result of applying a second cushion foundation with weak sustainability to the skin pore mimic, and NO3 represents the experimental result of applying a third cushion foundation with excellent sustainability to the skin pore mimic.

### DETAILED DESCRIPTION OF THE INVENTION

Various embodiments of the present document and the terms used in the embodiments are not intended to limit the technical features disclosed in the present document to the particular embodiments and should be understood as including various alterations, equivalents, or alternatives of the corresponding embodiments.

The inventors developed a skin mimic that has hydrophilicity and/or elasticity similar to human skin, and in which sebum components may move from the bottom to the top through capillary action.

One aspect of the present disclosure provides a skin pore mimic including a polydimethylsiloxane (PDMS) layer that includes polydimethylsiloxane (PDMS) and has a contact angle with water ranging from 90° to 110°, in which the PDMS layer includes a plurality of holes that penetrate through the upper surface of the PDMS layer and the lower surface of the PDMS layer.

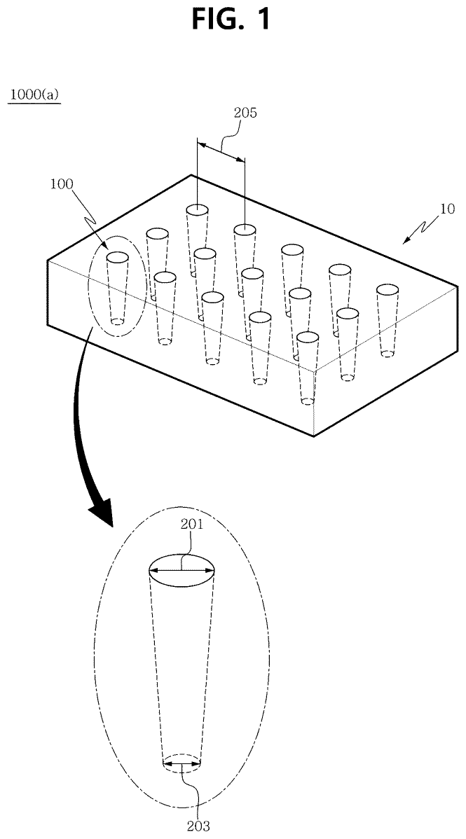

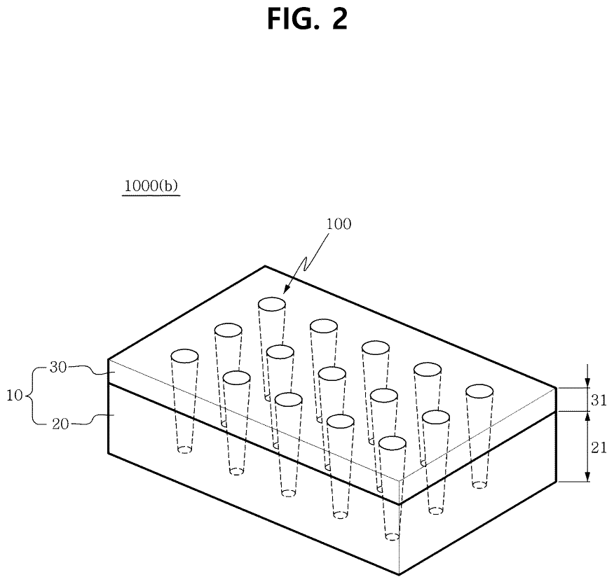

With reference to FIG. 1 to FIG. 2 below, a skin pore mimic 1000(a) or 1000(b) will be described. FIG. 1 to FIG. 2 are exemplary drawings for description purposes only, and the skin pore mimic according to one aspect of the present disclosure is not limited to the form or structure disclosed in the drawings.

The term “skin mimic (or skin equivalent)” may refer to a structure that reconstructs actual skin in three dimensions. The skin mimic may exhibit structural and/or functional characteristics similar to actual skin. The skin mimic is also referred to as artificial skin.

The term “skin pore mimic (or skin-mimicking substrate or skin pore equivalent)” may refer to a structure that mimics skin including pores in three dimensions. The skin pore mimic may simulate the form in which actual sebum is secreted through pores, and it may simulate the form in which actual sebum is secreted without the need for external power.

In an embodiment, the skin pore mimic 1000(a) or 1000(b) includes a PDMS layer 10 that includes polydimethylsiloxane and has a contact angle with water ranging from 90° to 110°. The PDMS layer 10 may include a plurality of holes 100 that penetrate through the upper surface of the PDMS layer 10 and the lower surface of the PDMS layer 10.

In an embodiment, the PDMS layer 10 may include a composition that includes PDMS. In an embodiment, the PDMS layer 10 may be made from a composition that includes PDMS.

The “contact angle” may refer to an angle at which a liquid forms a thermodynamic equilibrium on a solid surface. For example, when a liquid exists on a solid surface in air, among the angle formed between the tangent line at the contact point of the three phases, solid, liquid, and gas, and the solid surface, an angle on the side that includes the liquid, may be a contact angle of the liquid with respect to the solid. By determining the contact angle of a given material with respect to water, the hydrophilicity of the given material may be determined.

In an embodiment, the contact angle measurement may be converted to the surface energy of the solid using Young's equation, but this is not limited thereto.

In an embodiment, the surface energy of the PDMS layer 10, according to one aspect, may be determined by dropping a certain amount of water onto the PDMS layer 10 and measuring the contact angle. Here, only the contact angle comparison was made.

In an embodiment, the PDMS layer 10 may have a contact angle with water of 80° or more, 85° or more, 90° or more, 95° or more, 100° or more, 105° or more, 110° or more, 115° or more, 120° or more, 125° or more, 130° or more, 80° or less, 85° or less, 90° or less, 95° or less, 100° or less, 105° or less, 110° or less, 115° or less, 120° or less, 125° or less, 130° or less, or a combination range thereof (e.g., 90° to) 110°.

In an embodiment, the PDMS layer 10 may have a contact angle with water ranging from 80° to 130°, 90° to 130°, 90° to 120°, or 90° to 110°. The contact angles within the above ranges may be similar to the contact angle between actual human skin and water.

One aspect of the present disclosure may provide a skin pore mimic or PDMS layer 10 that exhibits hydrophilicity (surface energy) similar to that of human skin. One aspect of the present disclosure may provide a skin pore mimic or PDMS layer 10 that more closely implements the state in which sebum rises from actual human pores. Additionally, the skin pore mimic or PDMS layer 10 according to one aspect of the present disclosure may effectively adhere to the test material on its surface. This may be attributed to the fact that the skin pore mimic or PDMS layer 10 according to one aspect of the present disclosure has hydrophilicity (surface energy) similar to that of actual human skin.

In an embodiment, the upper surface of the PDMS layer 10 refers to the top surface of the PDMS layer 10, which may be the surface onto which the test material is applied. In an embodiment, the lower surface of the PDMS layer 10 refers to the bottom surface of the PDMS layer 10, which may be the surface in contact with artificial sebum or an oily solution.

In an embodiment, sebum components and/or oils may rise from the lower surface of the PDMS layer 10 to the upper surface of the PDMS layer 10 through the holes 100. In an embodiment, sebum components and/or oils may rise from the lower surface of the PDMS layer 10 to the upper surface of the PDMS layer 10 through the plurality of holes 100 included in the PDMS layer 10. Sebum components and/or oils may rise from the lower surface of the PDMS layer 10 to the upper surface of the PDMS layer 10 through capillary action.

Sebum components and/or oils may rise from the lower surface of the PDMS layer 10 to the upper surface of the PDMS layer 10 through the holes 100, influenced by the hydrophilicity of the PDMS layer 10. Sebum components and/or oils may rise to the upper surface of the PDMS layer 10 without external power, influenced by the hydrophilicity of the PDMS layer 10, and may rise uniformly to the upper surface of the PDMS layer 10 through the plurality of holes 100. Therefore, by using the skin pore mimic according to one aspect of the present disclosure, the sustainability with respect to sebum may be evaluated more objectively and accurately.

In an embodiment, the sustainability with respect to sebum of a test material (e.g., a cosmetic composition) may refer to the sustainability of the test material (e.g., a cosmetic composition) in the presence of sebum. For example, by using the skin pore mimic according to one aspect of the present disclosure, the sustainability of a test material (e.g., a cosmetic composition) in the presence of sebum may be evaluated more objectively and accurately.

The holes 100 may simulate the shape of actual human pores, where the cross-section of the holes becomes narrower as it moves from the epidermis to the dermis. The holes 100, having a shape where the area of the vertical cross-section increases as it moves upward, may facilitate the movement of sebum components present on the lower side to the upper side.

In an embodiment, the area of a first vertical cross-section of the hole 100 in the gravitational direction may be larger than the area of a second vertical cross-section of the hole in the gravitational direction, where the second vertical cross-section exists below the first vertical cross-section.

In an embodiment, the first vertical cross-section may be closer to the upper surface of the PDMS layer 10 than the second vertical cross-section. In an embodiment, the second vertical cross-section may be closer to the lower surface of the PDMS layer 10 than the first vertical cross-section.

The “vertical cross-section” may refer to a cross-section formed when the hole is cut by a plane perpendicular to the side of the hole. The “vertical cross-section” of the hole may refer to a cross-section formed when the hole is cut in a direction perpendicular to the gravitational direction.

In an embodiment, the first vertical cross-section may be the upper end cross-section of the hole 100. In an embodiment, the second vertical cross-section may be the lower end cross-section of the hole 100.

In an embodiment, the area of the upper end cross-section of the hole 100 may be larger than the area of the lower end cross-section of the hole 100.

The “upper end” may refer to the topmost end portion. The upper end of the hole 100 may be positioned on the upper surface of the PDMS layer 10. The “lower end” may refer to the bottommost end portion. The lower end of the hole 100 may be positioned on the lower surface of the PDMS layer 10.

In an embodiment, the ratio of the area of the upper end cross-section of the hole 100 to the area of the lower end cross-section of the hole 100 may be 3:1 to 7:1, 4:1 to 6:1, or 5:1 to 5.5:1, but is not limited thereto. For example, the ratio of the area of the upper end cross-section of the hole 100 to the area of the lower end cross-section of the hole 100 may be 3:1, 3.5:1, 4:1, 4.5:1, 5:1, 5.2:1, 5.5:1, 6:1, 6.5:1, 7:1, or a combination thereof, but is not limited thereto.

In an embodiment, the upper end cross-section and the lower end cross-section of the hole 100 may be circular.

In an embodiment, the ratio of a diameter 201 of the upper end cross-section of the hole 100 to a diameter 203 of the lower end cross-section of the hole 100 may be 1.5:1 to 5:1, 2:1 to 5:1, 2:1 to 4:1, or 2:1 to 3:1, but is not limited thereto. For example, the ratio of the diameter 201 of the upper end cross-section of the hole 100 to the diameter 203 of the lower end cross-section of the hole 100 may be 1.5:1, 2:1, 3:1, 4:1, 5:1, or a combination thereof, but is not limited thereto (see FIG. 1).

In an embodiment, the hole 100 may have a frustum shape, where the apex of the cone is cut in a direction parallel to the bottom surface. In an embodiment, the hole 100 may have a truncated cone shape.

In an embodiment, the PDMS layer 10 may include a surfactant with an HLB of 7 or more. By including a surfactant with an HLB of 7 or more, the hydrophilicity of the PDMS layer 10 may be implemented to be similar to that of actual skin. In an embodiment, the surfactant with an HLB of 7 or more may be a silicone-based surfactant.

In an embodiment, the PDMS layer 10 may include a silicone-based surfactant.

In an embodiment, the silicone-based surfactant may be a nonionic silicone-based surfactant. In an embodiment, the silicone-based surfactant may be trisiloxane ethoxylate. In an embodiment, the silicone-based surfactant may be Silwet L-77.

A distance 205 between the plurality of holes 100 may refer to a distance between the center of the upper end cross-section of a first hole and the center of the upper end cross-section of a second hole (see FIG. 1).

In an embodiment, the distance 205 between the plurality of holes 100 may be 0.4 to 1.6 mm, 0.5 to 1.5 mm, 0.6 to 1.4 mm, 0.7 to 1.3 mm, 0.8 to 1.2 mm, 0.9 to 1.1 mm, or 1 mm, but is not limited thereto. For example, the distance 205 between the plurality of holes 100 may be in a range of 0.4 mm or more, 0.5 mm or more, 0.6 mm or more, 0.7 mm or more, 0.8 mm or more, 0.9 mm or more, 1 mm or more, 1.1 mm or more, 1.2 mm or more, 1.3 mm or more, 1.4 mm or more, 1.5 mm or more, 1.6 mm or more, 0.4 mm or less, 0.5 mm or less, 0.6 mm or less, 0.7 mm or less, 0.8 mm or less, 0.9 mm or less, 1 mm or less, 1.2 mm or less, 1.3 mm or less, 1.4 mm or less, 1.5 mm or less, or a combination thereof, but is not limited thereto.

In an embodiment, the distance 205 between the plurality of holes 100 may be 2 to 8 times, 3 to 7 times, 4 to 6 times, or 5 times the diameter 201 of the upper end cross-section of a single hole 100, but is not limited thereto. For example, the distance 205 between the plurality of holes 100 may be in a range of 2 times or more, 3 times or more, 4 times or more, 5 times or more, 6 times or more, 7 times or more, 8 times or more, 2 times or less, 3 times or less, 4 times or less, 5 times or less, 6 times or less, 7 times or less, 8 times or less, or a combination thereof, but is not limited thereto.

In an embodiment, the hole 100 may be formed by etching a PDMS layer that does not include the hole, using optical or mechanical methods. In an embodiment, the hole 100 may be formed by pouring a PDMS mixture (e.g., a mixture of PDMS base and curing agent) into a negative mold, curing it, and then etching using optical and/or mechanical methods to create conical holes with increasing diameters towards the top, positioned at regular intervals.

In an embodiment, the hole 100 may be formed by pouring a PDMS mixture (e.g., a mixture of PDMS base and curing agent) into a mold including at least one reverse conical structure, where the diameter increases towards the top, and then curing the mixture.

In an embodiment, the PDMS layer 10 may be a single layer. In this case, the Young's modulus of the PDMS layer 10 may be at least 100 kPa but less than 300 kPa, or at least 150 kPa but less than 250 kPa. For example, the Young's modulus of the PDMS layer 10 may be at least 100 kPa, at least 150 kPa, at least 200 kPa, at least 250 kPa, less than 300 kPa, less than 250 kPa, less than 200 kPa, less than 150 kPa, or a combination of these ranges (e.g., 150 kPa to 250 kPa), but is not limited thereto.

When the PDMS layer 10 is made of a single layer with a Young's modulus within the above range, it may not simulate the elasticity of human skin, but it may implement a skin pore mimic that simulates the phenomenon of sebum rising. Using the corresponding skin pore mimic, the sustainability evaluation of sebum may be performed. Meanwhile, when the PDMS layer 10 is made of a single layer with a Young's modulus lower than the above range (e.g., less than 100 kPa, or 20 to 40 kPa), the surface of the PDMS layer 10 may become excessively sticky, causing the contact angle to differ from that of actual skin. This may present a problem in positioning the test material on the mimic.

The term “Young's modulus” may refer to the elastic property of a solid, and it is also known as the “modulus of elasticity” or “elastic modulus.” Young's modulus refers to the ratio of the pressure (stress) applied to an object to the deformation of the object. Young's modulus is a modulus of elasticity that indicates the degree of deformation of an object when pressure is applied. The basic principle of measuring Young's modulus is based on the property of undergoing elastic deformation when an object is compressed or stretched, and returning to their original shape once the load is removed.

In an embodiment, Young's modulus may be measured using a universal tensile machine. In an embodiment, Young's modulus may be measured at room temperature or at 37° C. (the temperature of human skin).

In an embodiment, the PDMS layer 10 may include two or more layers. In an embodiment, the PDMS layer 10 may include two layers. In an embodiment, the PDMS layer 10 may be composed of two layers.

In an embodiment, the PDMS layer 10 includes a first layer 20 and a second layer 30 stacked on the upper side of the first layer 20, where the Young's modulus of the second layer 30 may be higher than the Young's modulus of the first layer 20.

By stacking the second layer 30, which has a higher Young's modulus than the first layer 20, on the upper side of the first layer 20, the stickiness of the upper surface may be minimized. By stacking the second layer 30, which has a higher Young's modulus than the first layer 20, on the upper side of the first layer 20, the surface properties and elasticity of actual skin may be more closely simulated. The second layer 30 may simulate the epidermis, and the first layer 20 may simulate the dermis.

The skin pore mimic, which includes the PDMS layer 10 including the first layer 20 and the second layer 30 stacked on the upper side of the first layer 20, where the Young's modulus of the second layer 30 is higher than that of the first layer 20, may exhibit elasticity more similar to the structure of actual skin.

When the PDMS layer 10 includes a first layer and a second layer with the characteristics disclosed in this specification, it may simulate the elasticity of human skin while simultaneously simulating the phenomenon of sebum rising, thus implementing the skin pore mimic. By using the skin pore mimic with these characteristics, not only the sustainability of the test material with respect to sebum but also its sustainability under external forces (e.g., movement) may be evaluated.

In an embodiment, the Young's modulus of the first layer 20 may be less than 100 kPa. For example, the Young's modulus of the first layer 20 may be less than 100 kPa, less than 90 kPa, less than 80 kPa, less than 70 kPa, less than 60 kPa, less than 50 kPa, less than 40 kPa, or less than 30 kPa, but is not limited thereto.

In an embodiment, the Young's modulus of the second layer 30 may be at least 100 kPa but less than 300 kPa, or at least 150 kPa but less than 250 kPa. For example, the Young's modulus of the second layer 30 may be at least 100 kPa, at least 150 kPa, at least 200 kPa, at least 250 kPa, less than 300 kPa, less than 250 kPa, less than 200 kPa, less than 150 kPa, or a combination of these ranges, but is not limited thereto.

The above-mentioned Young's modulus may be measured at room temperature or at 37° C. (the temperature of human skin).

In an embodiment, the first layer 20 may include a PDMS base and a curing agent in a weight ratio of 40:1. In an embodiment, the first layer 20 may be made from a PDMS solution in which a PDMS base and a curing agent are mixed in a weight ratio of 40:1. The curing agent may be the Sylgard 184 silicone elastomer curing agent.

In an embodiment, the second layer 30 may include a PDMS base and a curing agent in a weight ratio of 20:1. In an embodiment, the second layer 30 may be made from a PDMS solution formed by mixing a PDMS base and a curing agent in a weight ratio of 20:1. The curing agent may be the Sylgard 184 silicone elastomer curing agent.

In an embodiment, the thickness of the second layer 30 may be thinner than the thickness of the first layer 20.

In an embodiment, the ratio of the thickness of the second layer 30 to the thickness of the first layer 20 may be 1:1.5 to 1:5, 1:2 to 1:4, or 1:3, but is not limited thereto. For example, the ratio of the thickness of the second layer 30 to the thickness of the first layer 20 may be 1:1.5 or more, 1:2 or more, 1:3 or more, 1:4 or more, 1:5 or more, 1:2 or less, 1:3 or less, 1:4 or less, 1:5 or less, or a combination of these ranges, but is not limited thereto.

One aspect of the present disclosure provides a skin pore mimic that allows sebum components to rise uniformly to the surface under conditions similar to actual human skin (e.g., hydrophilicity, elasticity, etc.). By using this, the sustainability of the test material under conditions similar to those of actual skin may be evaluated more objectively and accurately. Meanwhile, in the related art, i) the sustainability of a cosmetic film with respect to sebum was evaluated by applying sebum components to artificial skin and then placing the cosmetic film over the applied sebum, or ii) the sustainability of a cosmetic film with respect to sebum was evaluated by placing the cosmetic film on the surface of a porous material and allowing the sebum components to pass through the porous material. These methods do not implement the phenomenon of sebum rising under conditions similar to actual skin, making it difficult to perform an objective evaluation.

In an embodiment, the skin pore mimic 1000(a) or 1000(b) may be used for evaluating the sustainability with respect to at least one of sebum or external forces of the test material. In an embodiment, using the skin pore mimic 1000(a) that includes a single PDMS layer, the sustainability of a test material with respect to sebum may be evaluated. In an embodiment, using a skin pore mimic 1000(b) that includes a PDMS layer with two layers of differing Young's modulus, the sustainability of a test material with respect to sebum may be evaluated, while simultaneously evaluating the sustainability of the test material with respect to external forces.

In an embodiment, the sustainability of a test material (e.g., a cosmetic composition) with respect to external forces may refer to the sustainability of the test material (e.g., a cosmetic composition) in environment under external force. For example, by using the skin pore mimic according to one aspect of the present disclosure, the sustainability of a test material (e.g., a cosmetic composition) in environment under external force may be evaluated more objectively and accurately.

In an embodiment, the test material may be at least one of a cosmetic composition or a cosmetic ingredient.

In an embodiment, the cosmetic composition may be at least one form selected from the group consisting of liquid, powder, and solid, but is not limited thereto.

In an embodiment, the cosmetic composition may be at least one selected from the group consisting of foundation, cushion, sunscreen, and powder (pigments), but is not limited thereto. In an embodiment, the cosmetic composition may be a cosmetic composition that includes an excess amount of powder (pigments).

Additionally, one aspect of the present disclosure provides a use for evaluating the sustainability of a test material with respect to at least one of sebum or external forces using the aforementioned skin pore mimic.

Additionally, one aspect of the present disclosure provides a use of the skin pore mimic for evaluating the sustainability of a test material with respect to at least one of sebum or external forces.

As the skin pore mimic and test material have been described above, a detailed description is omitted.

In addition, one aspect of the present disclosure provides a method of evaluating the sustainability of a test material, including: applying a test material to an upper surface of the PDMS layer of the skin pore mimic; and contacting a lower surface of the PDMS layer with artificial sebum.

As the skin pore mimic, PDMS layer, and test material have been described above, a detailed description is omitted.

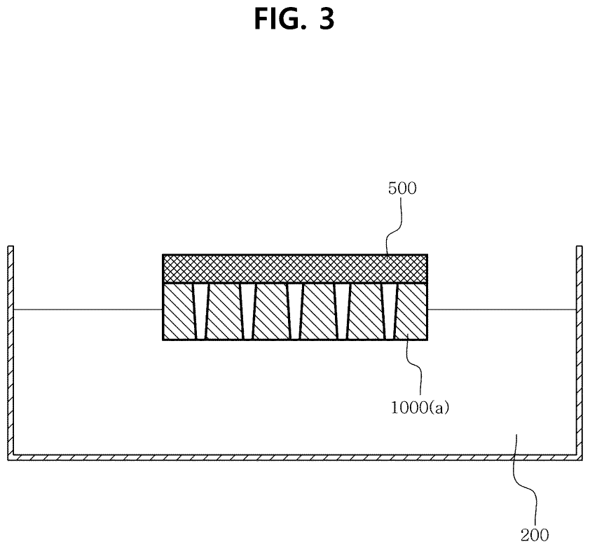

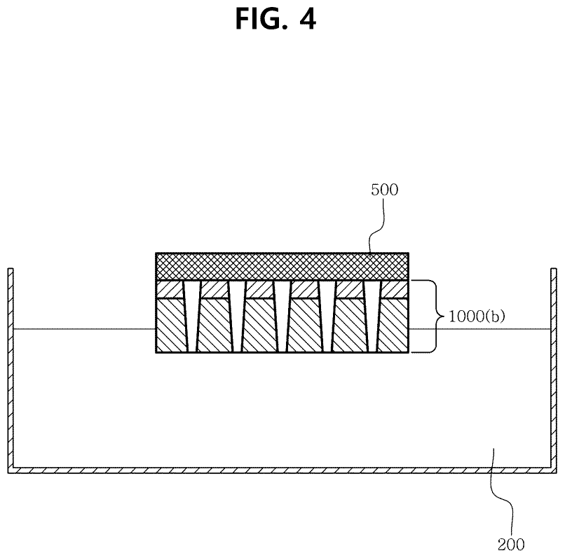

With reference to FIG. 3 to FIG. 4 below, the method of evaluating the sustainability of a test material using the skin pore mimic 1000(a) or 1000(b) will be described. FIG. 3 to FIG. 4 are exemplary drawings for description purposes only, and the method of evaluating the sustainability of a test material according to one aspect of the present disclosure is not limited to the form or structure disclosed in the drawings.

In an embodiment, the method of evaluating the sustainability of a test material includes: applying a test material 500 to an upper surface of the PDMS layer of the skin pore mimic 1000(a) or 1000(b); and contacting a lower surface of the PDMS layer with artificial sebum 200.

The contacting of the lower surface of the PDMS layer with the artificial sebum 200 may be performed either before or after applying the test material 500 to the upper surface of the PDMS layer.

The applying of the test material 500 to the upper surface of the PDMS layer may be performed either before or after contacting the lower surface of the PDMS layer with the artificial sebum 200.

The applying of the test material 500 to the upper surface of the PDMS layer and the contacting of the lower surface of the PDMS layer with the artificial sebum 200 may be performed simultaneously.

In an embodiment, the applying of the test material 500 to the PDMS layer may be applying the test material 500 to at least a portion of the upper surface of the PDMS layer of the skin pore mimic 1000(a) or 1000(b).

The artificial sebum 200 is a material provided to the skin pore mimic for evaluating sustainability with respect to sebum, and it may be a material that simulates the oils and/or sebum secreted from the skin.

In an embodiment, the artificial sebum 200 may be a material separated from the human body or a synthetic material.

In an embodiment, the artificial sebum 200 may include a material separated from the human body (e.g., sebum).

In an embodiment, the artificial sebum 200 may be made using publicly known methods, and the specific components are not limited. For example, the artificial sebum 200 may include at least one selected from the group consisting of squalene, wax monoesters, caprylic/capric triglycerides, fatty acids, oleic acid, and vitamin E (P W Wertz. 2009, Human synthetic sebum formulation and stability under conditions of use and storage, Int J Cosmet Sci. Feb. 2009;31(1):21-5).

In an embodiment, the artificial sebum 200 may be at least one of a liquid phase or a solid phase, but is not limited thereto.

In an embodiment, the contacting of the artificial sebum 200 may be contacting at least a portion of the lower surface of the PDMS layer of the skin pore mimic 1000(a) or 1000(b) with the artificial sebum 200.

In an embodiment, the artificial sebum 200 may be included in any container.

In an embodiment, the method of evaluating the sustainability of a test material may further include: analyzing the degree of deformation of the applied test material 500 to evaluate the sustainability of the test material with respect to sebum.

The test material may be applied to the upper surface of the PDMS layer of the skin pore mimic 1000(a) or 1000(b) to form a film of the test material. In this case, by analyzing the degree of deformation of the film of the test material, the sustainability of the test material with respect to sebum may be evaluated.

In an embodiment, the method of evaluating the sustainability of a test material may further include: applying an external force to the skin pore mimic 1000(a) or 1000(b) and analyzing the degree of deformation of the applied test material 500 to evaluate the sustainability of the test material with respect to the external force.

In an embodiment, the method of evaluating the sustainability of a test material may be a method of evaluating the sustainability of the test material on the skin.

In an embodiment, the sustainability may refer to sustainability with respect to at least one of oils or external forces.

Hereinafter, the present invention will be described in detail with reference to Examples to specifically describe the present invention. The following examples are provided solely for illustrative purposes to aid in understanding the present invention, and are not intended to limit the scope and range of the present invention.

### 1. Manufacture of Skin Pore Mimic

### (1) Example 1—Manufacture of Skin Pore Mimic Having Two PDMS Layers

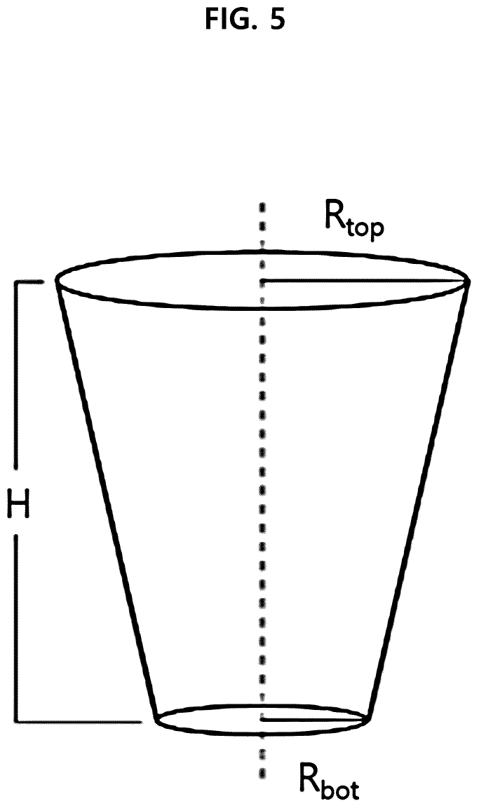

A polydimethylsiloxane (PDMS)-based skin pore mimic was manufactured, which has elasticity similar to actual skin and a surface hydrophilicity that allows sebum to pass easily through the pores. Additionally, to exhibit a mechanism similar to the actual pore shape, the skin pore mimic was designed to have conical-shaped holes (hole that is narrow at the bottom and wide at the top) (see FIG. 2 and FIG. 5). The inventors used a PDMS polymer base and curing agent in a 40:1 ratio to implement the elasticity similar to that of actual human skin. Additionally, to implement the surface hydrophilicity similar to actual human skin, a PDMS mixture of PDMS polymer base and curing agent in a 20:1 ratio was applied over the 40:1 PDMS curing layer, and then thermally cured to manufacture a skin pore mimic with two PDMS layers.

Specifically, a first PDMS solution (PDMS polymer base and curing agent in a 40:1 weight ratio; Young's modulus approximately 30 kPa) and a second PDMS solution (PDMS polymer base and curing agent in a 20:1 weight ratio; Young's modulus approximately 200 kPa) were prepared using the SYLGARDTM 184 silicone elastomer kit (Dow, USA). The first PDMS solution and the surfactant (Silwet L-77) were mixed in a weight ratio of 100:0.13 to prepare a first mixture, and the second PDMS solution and the surfactant (Silwet L-77) were mixed in a weight ratio of 100:0.13 to prepare a second mixture. The first mixture was poured into a negative square mold and cured at 65° C. for 5 hours to obtain the PDMS layer. In this case, the first mixture was poured to a height of ¾ of the mold. Then, the second mixture was poured into the remaining area of the mold on top of the cured PDMS first layer, filling the remaining area of the mold, and it was cured at 65° C. for 5 hours to stack the second layer (20:1 PDMS curing layer) on top of the first layer. After curing, the two PDMS layers were etched using optical or mechanical methods to create conical holes, with the diameter increasing towards the top (lower diameter 100 μm, upper diameter 200 μm), positioned at regular intervals (1 mm apart). Afterward, the cured two-layer PDMS was gently peeled off from the mold, obtaining a skin pore mimic (Example 1) with two PDMS layers. Meanwhile, a mimic with two PDMS layers including holes with increasing diameters towards the top may also be manufactured by creating a reverse conical structure mold (lower diameter 100 μm, upper diameter 200 μm) and pouring the PDMS mixture into the mold to cure it.

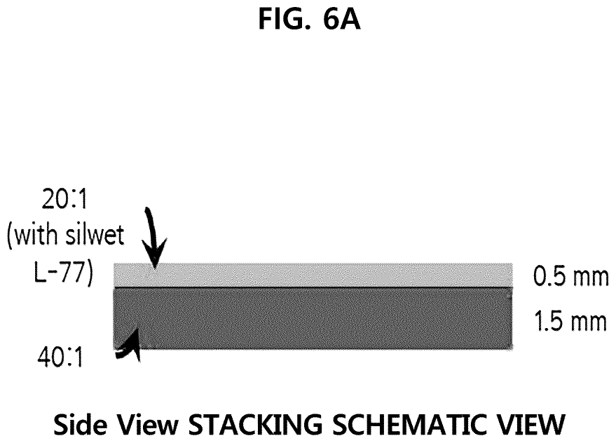

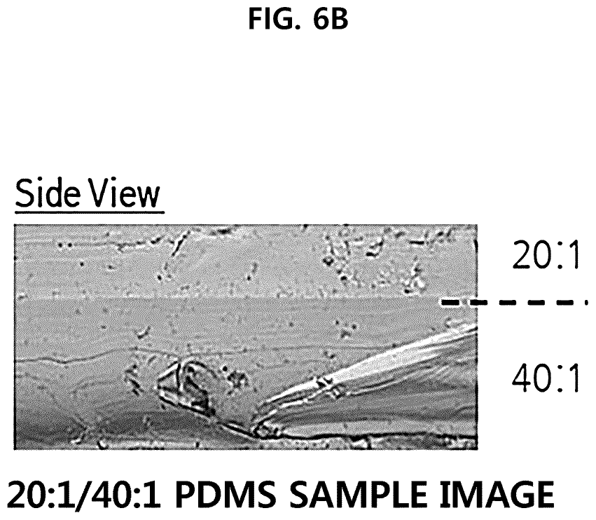

The 40:1 PDMS curing layer, which is the lower layer, exhibited properties similar to the elasticity of actual skin. By using a PDMS mixture of a PDMS polymer base and a curing agent mixed at a ratio of 20:1, which has a different elasticity than the lower layer, with a thickness three times thinner than the lower layer, the upper layer was formed. As a result, the overall elasticity of the skin pore mimic did not significantly differ from that of the 40:1 PDMS curing layer, while achieving a hydrophilic surface and a non-sticky surface. FIG. 6A to FIG. 6B illustrate schematic views and images showing the side view of the skin pore mimic (Example 1) with two PDMS layers.

### (2) Example 2—Manufacture of Skin Pore Mimic Having Single PDMS Layer

A polydimethylsiloxane (PDMS)-based skin pore mimic was manufactured to have a surface hydrophilicity that allows sebum to pass easily through the pores and to exhibit a mechanism similar to the actual pore shape, with conical-shaped holes (hole that is narrow at the bottom and wide at the top) (see FIG. 1 and FIG. 5). Specifically, a first PDMS solution (PDMS polymer base and curing agent in a 20:1 weight ratio; Young's modulus approximately 200 kPa) was prepared using the SYLGARDTM 184 silicone elastomer kit (Dow, USA). The first PDMS solution was mixed with a surfactant (Silwet L-77) in a 100:0.13 weight ratio. After mixing, the mixture was poured into a negative square mold and cured at 65° C. for 5 hours to obtain a PDMS layer. In this case, the mixture was poured to completely fill the mold. After curing, the PDMS layer was etched using optical or mechanical methods to create conical holes, with the diameter increasing towards the top (lower diameter 100 μm, upper diameter 200 μm), positioned at regular intervals (1 mm apart). Afterward, the cured PDMS layer was gently peeled off from the mold, obtaining a skin pore mimic (Example 2) with a single PDMS layer. Meanwhile, a mimic with a single PDMS layer including holes with increasing diameters towards the top may also be manufactured by creating a reverse conical structure mold (lower diameter 100 μm, upper diameter 200 μm) and pouring the PDMS mixture into the mold to cure it.

### 2. Performance Evaluation of Skin Pore Mimic

### (1) Verification of Properties of Skin Pore Mimic

To verify whether the skin pore mimic has hydrophilicity similar to that of actual human skin, a contact angle, commonly used for this purpose, was measured. Specifically, after dropping 2 to 5 μL of water onto the upper surface of the skin pore mimic of Example 1, the contact angle was measured after a predetermined amount of time had passed.

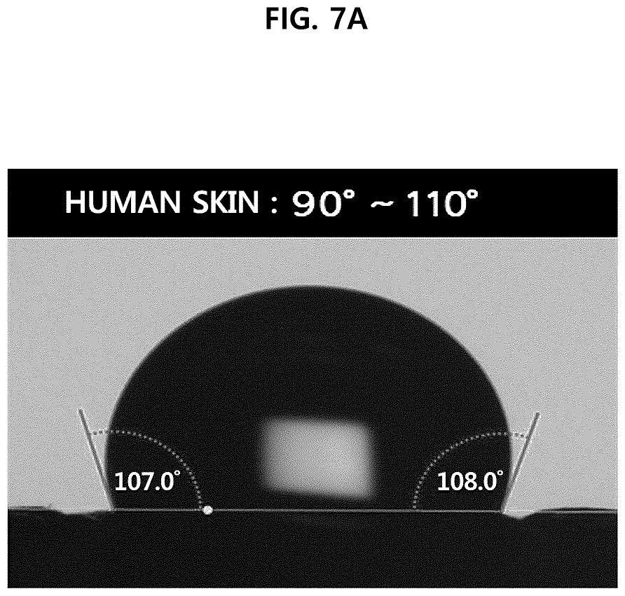

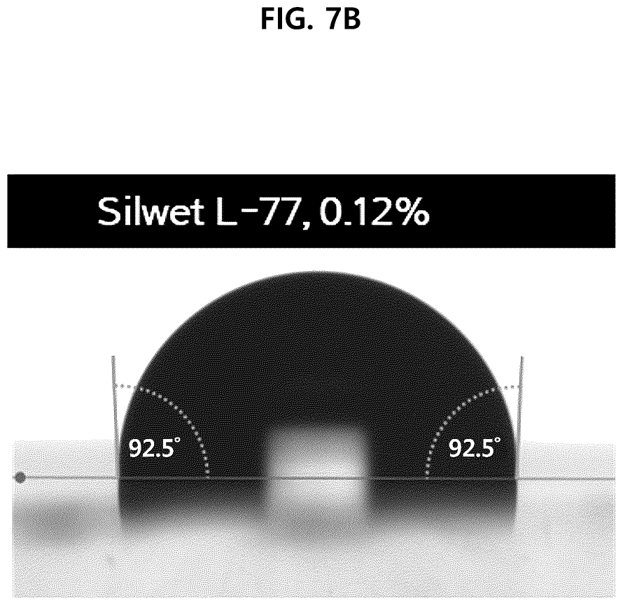

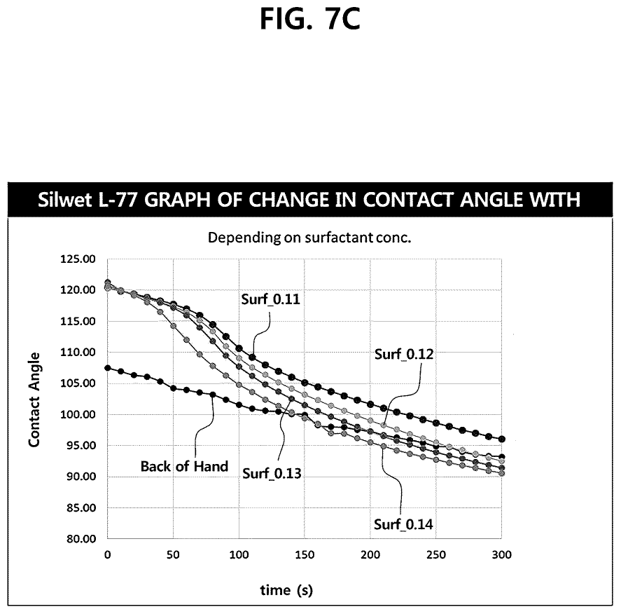

As a result, the skin pore mimic of the present disclosure exhibited a contact angle with water in the range of 90 to 110°, which is similar to the contact angle between water and the surface of actual human skin (see FIG. 7A to FIG. 7C).

### (2) Verification of Sebum Secretion Performance of Skin Pore Mimic

The ability of the skin pore mimic to accurately simulate the manner in which actual skin secretes sebum was verified. Specifically, a portion of the skin pore mimic of Example 1 was immersed in a container including artificial sebum (squalene 124 mg, wax monoester (jojoba oil) 250 mg, caprylic/capric triglyceride 447 mg, oleic acid 170 mg, vitamin E±10 mg), ensuring that the lower surface of the skin pore mimic (the surface with smaller hole diameters) came into contact with the solution including the sebum. After 5 to 10 minutes, the condition of the upper surface of the skin pore mimic (the surface with larger hole diameters) was checked.

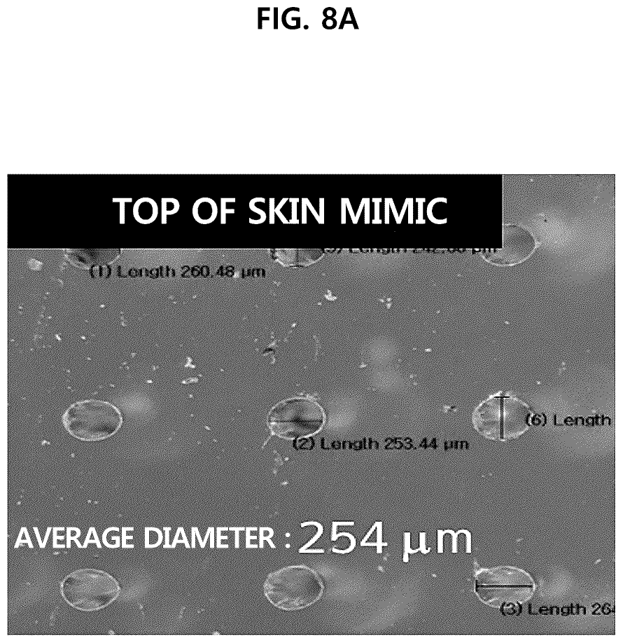

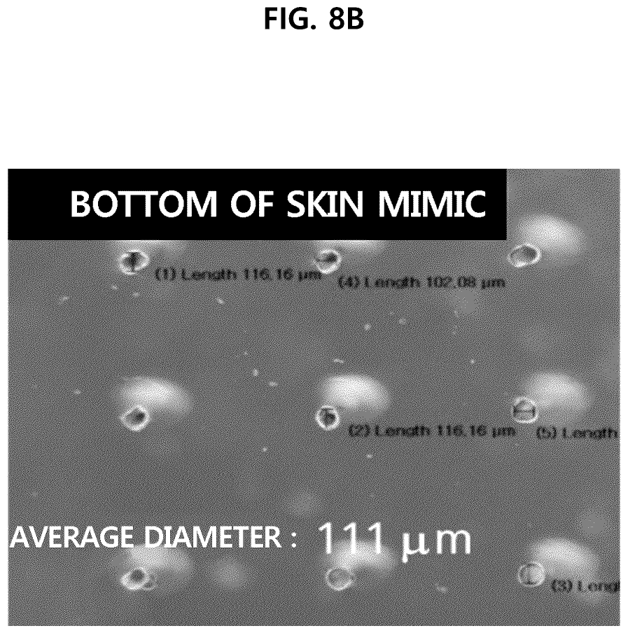

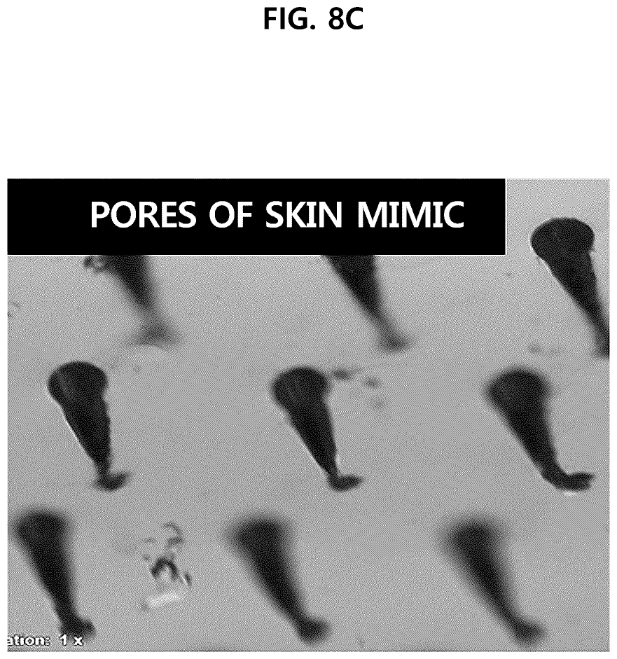

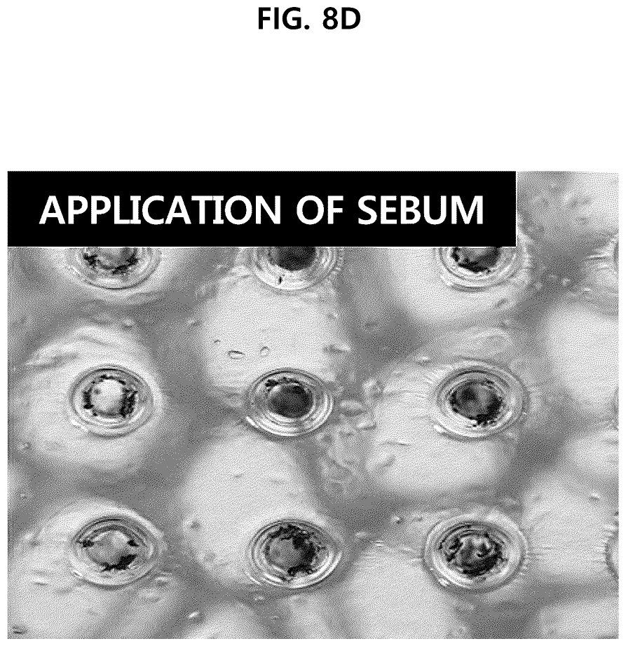

As a result, the sebum was successfully transferred through the holes from the bottom to the upper surface of the PDMS, and the upper surface of the skin pore mimic (the surface with larger hole diameters) exhibited a wetting state (see FIG. 8D). FIG. 8A to FIG. 8C illustrate the skin pore mimic before the application of artificial sebum.

### (3) Sustainability Evaluation Using Skin Pore Mimic

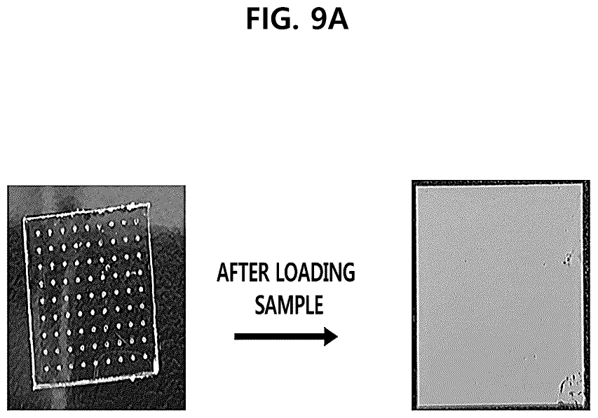

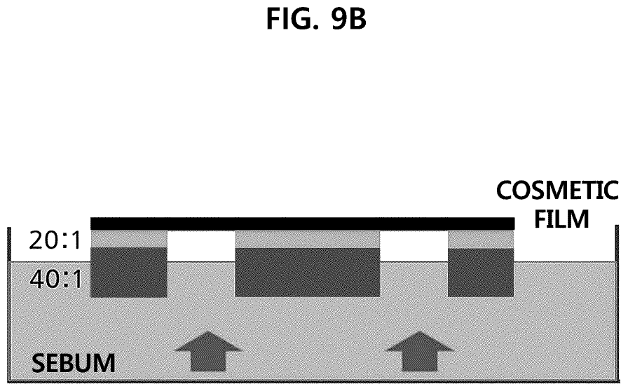

The ability of the skin pore mimic to evaluate the sustainability of a cosmetic composition with respect to sebum was verified. Specifically, three skin pore mimics of Example 1 were prepared, and three commercially available cushion foundations were purchased to serve as the test materials: i) a first cushion foundation (NO1), ii) a second cushion foundation (NO2), and iii) a third cushion foundation (NO3). These were uniformly applied to the upper surface of each mimic using a spin coater (see FIG. 9A). In this case, the first and second cushion foundations of (i) and (ii) were products with weak sustainability, while the third cushion foundation of (iii) was a product with excellent sustainability. The skin pore mimic with the test material applied was placed in a bath including artificial sebum (see FIG. 9B), and the sustainability of the test material with respect to sebum was verified.

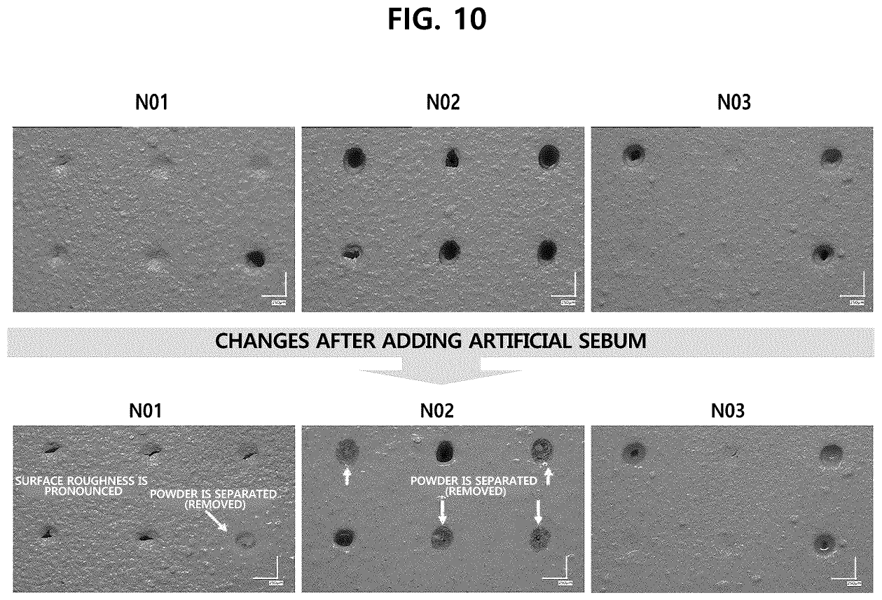

As a result, the third cushion foundation with excellent sustainability showed almost no change in the applied form on the mimic even after the application of artificial sebum (first column from the right in FIG. 10). Meanwhile, the first and second cushion foundations with weak sustainability showed changes in the applied form on the mimic after the application of artificial sebum. Specifically, after the application of artificial sebum, the first cushion foundation showed that the powders were separated from the surface by the sebum, and the surface roughness became more pronounced. Additionally, after the application of artificial sebum, the powders in the second cushion foundation were separated from the surface by the sebum (first and second columns from the left in FIG. 10). Using the skin pore mimic of the present invention, it was confirmed not only the changes in the cosmetic film caused by sebum but also changes in the roughness of the cosmetic film and/or the breaking of the cosmetic film. Through these results, it was found that the skin pore mimic of the present invention may effectively evaluate the sustainability of any cosmetic formulation with respect to sebum.

### EMBODIMENTS

Embodiment 1: A skin pore mimic, comprising: a PDMS layer that includes polydimethylsiloxane (PDMS) and has a contact angle with water ranging from 90° to 110°, in which the PDMS layer includes a plurality of holes that penetrate through an upper surface of the PDMS layer and a lower surface of the PDMS layer.

Embodiment 2: In the skin pore mimic of Embodiment 1, an area of a first vertical cross-section of the hole in a gravitational direction is larger than an area of a second vertical cross-section of the hole in the gravitational direction, and the second vertical cross-section exists below the first vertical cross-section.

Embodiment 3: In the skin pore mimic of Embodiment 2, the first vertical cross-section is an upper end cross-section of the hole, and the second vertical cross-section is a lower end cross-section of the hole.

Embodiment 4: In the skin pore mimic of Embodiment 3, a ratio of an area of the upper end cross-section to an area of the lower end cross-section is 3:1 to 7:1.

Embodiment 5: In the skin pore mimic of any one of Embodiments 1 to 4, the hole has a frustum shape, where an apex of a cone is cut in a direction parallel to a bottom surface thereof.

Embodiment 6: In the skin pore mimic of any one of Embodiments 1 to 5, the PDMS layer includes a surfactant with an HLB of 7 or more.

Embodiment 7: In the skin pore mimic of any one of Embodiments 1 to 6, the PDMS layer includes a silicone-based surfactant.

Embodiment 8: In the skin pore mimic of any one of Embodiments 1 to 7, the PDMS layer includes: a first layer; and a second layer stacked on top of the first layer, where Young's modulus of the second layer is higher than that of the first layer.

Embodiment 9: In the skin pore mimic of Embodiment 8, the Young's modulus of the first layer at room temperature or 37° C. is less than 100 kPa.

Embodiment 10: In the skin pore mimic of Embodiment 8 or 9, the Young's modulus of the second layer at room temperature or 37° C. is at least 100 kPa but less than 300 kPa.

Embodiment 11: In the skin pore mimic of any one of Embodiments 8 to 10, a thickness of the second layer is thinner than a thickness of the first layer.

Embodiment 12: In the skin pore mimic of any one of Embodiments 1 to 11, the skin pore mimic is for evaluating sustainability of a test material with respect to at least one of sebum or external forces.

Embodiment 13: In the skin pore mimic of any one of Embodiments 1 to 12, the test material is at least one of a cosmetic composition or a cosmetic ingredient.

Embodiment 14: A method of evaluating sustainability of a test material, comprising: applying a test material to the upper surface of the PDMS layer of the skin pore mimic of any one of Embodiments 1 to 13; and contacting the lower surface of the PDMS layer with artificial sebum.

Embodiment 15: In the method of Embodiment 14, the method further includes: analyzing a degree of deformation of the applied test material to evaluate sustainability of the test material with respect to sebum.

Embodiment 16: In the method of Embodiment 14 or 15, the method further includes: applying an external force to the skin pore mimic, and analyzing a degree of deformation of the applied test material to evaluate sustainability of the test material with respect to the external force.

Embodiment 17: In the method of any one of Embodiments 14 to 16, the test material is at least one of a cosmetic composition or a cosmetic ingredient.

Embodiment 18: In the method of any one of Embodiments 14 to 17, the sustainability is sustainability with respect to at least one of oils or external forces.

### DESCRIPTION OF REFERENCE NUMERALS

1000(a) and 1000(b): Skin pore mimic
10: PDMS layer
20: PDMS first layer
30: PDMS second layer
21: Thickness of PDMS first layer
31: Thickness of PDMS second layer
100: Hole
201: Diameter of upper end cross-section of hole
203: Diameter of lower end cross-section of hole
205: Distance between first hole and second hole
200: Artificial sebum
500: Film of test material applied to upper surface of PDMS layer

We claim:

1. A skin pore mimic, comprising:
a PDMS layer including polydimethylsiloxane (PDMS) and having a contact angle with water ranging from 90° to 110°,
wherein the PDMS layer includes a plurality of holes that penetrate through an upper surface of the PDMS layer and a lower surface of the PDMS layer.

2. The skin pore mimic of claim 1, wherein an area of a first vertical cross-section of the hole in a gravitational direction is larger than an area of a second vertical cross-section of the hole in the gravitational direction, and the second vertical cross-section exists below the first vertical cross-section.

3. The skin pore mimic of claim 2, wherein the first vertical cross-section is an upper end cross-section of the hole, and the second vertical cross-section is a lower end cross-section of the hole.

4. The skin pore mimic of claim 3, wherein a ratio of an area of the upper end cross-section to an area of the lower end cross-section is 3:1 to 7:1.

5. The skin pore mimic of claim 2, wherein the hole has a frustum shape, where an apex of a cone is cut in a direction parallel to a bottom surface thereof.

6. The skin pore mimic of claim 1, wherein the PDMS layer includes a surfactant with an HLB of 7 or more.

7. The skin pore mimic of claim 1, wherein the PDMS layer includes a silicone-based surfactant.

8. The skin pore mimic of claim 1, wherein the PDMS layer includes:
a first layer; and
a second layer stacked on top of the first layer, and
wherein Young's modulus of the second layer is higher than Young's modulus of the first layer.

9. The skin pore mimic of claim 8, wherein the Young's modulus of the first layer at room temperature or 37° C. is less than 100 kPa.

10. The skin pore mimic of claim 8, wherein the Young's modulus of the second layer at room temperature or 37° C. is at least 100 kPa but less than 300 kPa.

11. The skin pore mimic of claim 8, wherein a thickness of the second layer is thinner than a thickness of the first layer.

12. The skin pore mimic of claim 1, wherein the skin pore mimic is for evaluating sustainability of a test material with respect to at least one of sebum or external forces.

13. The skin pore mimic of claim 12, wherein the test material is at least one of a cosmetic composition or a cosmetic ingredient.

14. A method of evaluating sustainability of a test material, comprising:
applying a test material to the upper surface of the PDMS layer of the skin pore mimic of claim 1; and
contacting the lower surface of the PDMS layer with artificial sebum.

15. The method of claim 14, further comprising:
analyzing a degree of deformation of the applied test material to evaluate sustainability of the test material with respect to sebum.

16. The method of claim 14, further comprising:
applying an external force to the skin pore mimic, and analyzing a degree of deformation of the applied test material to evaluate sustainability of the test material with respect to the external force.

17. The method of claim 14, wherein the test material is at least one of a cosmetic composition or a cosmetic ingredient.

18. The method of claim 14, wherein the sustainability is sustainability with respect to at least one of oils or external forces.
